(19) United States
(12) Patent Application Publication
(10) Pub. No.: US 2016/0206645 A1
(43) Pub. Date: Jul. 21, 2016

(54) COMBINATION THERAPIES FOR TREATMENT OF HCV

(71) Applicants: Janssen Pharmaceuticals, Inc., Titusville, NJ (US); Bristol Myers Squibb, Princeton, NJ (US)

(72) Inventors: Maria Gloria Beumont, Bievres (FR); Ann Tamara Vandevoorde, Beerse (BE); Leen Roger Itta Vijgen, Beerse (BE); Greta Irene A. Beets, Beerse (BE); Donghan Luo, Titusville, NJ (US); Sivi Mahadevan, Ravels (BE); Thomas Naoki Kakuda, South San Francisco, CA (US); Peter Josef Maria Van Remoortere, Princeton, NJ (US); Gaston Rafael Picchio, San Diego, CA (US); Bert Jacquemyn, Beerse (BE); Stephanie Noviello, Bridgewater, NJ (US); Eric A. Hughes, Skillman, NJ (US)

(21) Appl. No.: 15/001,705

(22) Filed: Jan. 20, 2016

Related U.S. Application Data
(60) Provisional application No. 62/105,442, filed on Jan. 20, 2015.
(60) Provisional application No. 62/127,436, filed on Mar. 3, 2015.

(51) Int. Cl. A61K 31/7072; A61K 31/4178; A61K 31/4709

(52) U.S. Cl. CPC A61K 31/7072; A61K 31/4709; A61K 31/4178

(57) ABSTRACT

The present disclosure is directed to the use of a combination of simeprevir, daclatasvir, and sofosbuvir for the treatment of hepatitis C virus infection.

### CROSS REFERENCE TO RELATED APPLICATION

This application claims the benefit of U.S. Provisional Application No. 62/105,442, filed Jan. 20, 2015, and U.S. Provisional Application No. 62/127,426, filed Mar. 3, 2015, the entireties of which are incorporated by reference herein.

### TECHNICAL FIELD

The present disclosure relates to the use of a combination of simeprevir, daclatasvir, and sofosbuvir for the treatment of hepatitis C virus infection.

### BACKGROUND

Hepatitis C virus (HCV), a member of the Flaviviridae family of viruses in the hepacivirus genus, is the leading cause of chronic liver disease worldwide. Although the development of diagnostics and blood screening has considerably reduced the rate of new infections, HCV remains a global health burden due to its chronic nature and its potential for long-term liver damage. There are six major HCV genotypes (1-6) and multiple subtypes (represented by letters). Genotype 1b is predominant in Europe, while genotype 1a is predominant in North America. Genotype is clinically important in determining potential response to therapy and the required duration of such therapy.

HCV is mainly transmitted by blood contact. Following initial acute infection, a majority of infected individuals develops chronic hepatitis because HCV replicates preferentially in hepatocytes but is not directly cytopathic. Over decades, a considerable number of infected persons develop fibrosis, cirrhosis and hepatocellular carcinoma, with chronic HCV infection being the leading cause for liver transplantation. This and the number of patients involved, has made HCV the focus of considerable medical research.

Replication of the genome of HCV is mediated by a number of enzymes, amongst which is HCV NS3/4A serine protease and its associated cofactor, NS4A. Other essential enzymes in this process are NSSB polymerase and NSSA. NS3/4A serine protease, NSSA and NSSB polymerase are considered to be essential for viral replication and inhibitors of these enzymes are considered drug candidates for HCV treatment.

It has now been found that a combination of three specific direct acting anti-virals provide alternative/improved HCV therapy, for example, reduces treatment time and improved treatment of HCV.

### SUMMARY

The present disclosure is directed to methods of treating HCV in a patient comprising administering to the patient an effective amount of:
simeprevir, or a pharmaceutically acceptable salt thereof;
daclastavir, or a pharmaceutically acceptable salt thereof; and
sofosbuvir, or a pharmaceutically acceptable salt thereof;
   wherein the administration terminates after a period of time that is 6, 7, 8, 9, 10, 11, or 12 weeks.

### BRIEF DESCRIPTION OF THE DRAWINGS

FIG. 1 depicts the on-treatment virologic response (HCV RNA<15 IU/mL undetectable) over time.

FIG. 2 depicts the mean exposure of simeprevir in Child-Pugh A and Child-Pugh B subjects at week 2 and week 8 of treatment with SMV+SOF+DCV.

FIG. 3A depicts the mean exposure of sofosbuvir in Child-Pugh A and Child-Pugh B subjects at week 2 and week 8 of treatment with SMV+SOF+DCV.

FIG. 3B depicts the mean exposure of GS-331007 (a sofosbuvir metabolite) in Child-Pugh A and Child-Pugh B subjects at week 2 and week 8 of treatment with SMV+SOF+DCV.

FIG. 4 depicts the mean exposure of daclastavir in Child-Pugh A and Child-Pugh B subjects at week 2 and week 8 of treatment with SMV+SOF+DCV.

FIG. 5 depicts the change from baseline to follow up at week 12 of treatment of hemoglobin in Child-Pugh A and Child-Pugh B subjects.

FIG. 6 depicts the change from baseline to follow up at week 12 of treatment of bilirubin in Child-Pugh A and Child-Pugh B subjects.

FIG. 7 depicts the change from baseline to follow up at week 12 of treatment of INR (international normalized ratio) in Child-Pugh A and Child-Pugh B subjects.

FIG. 8 depicts the change from baseline to follow up at week 12 of treatment of albumin in Child-Pugh A and Child-Pugh B subjects.

### DETAILED DESCRIPTION OF ILLUSTRATIVE EMBODIMENTS

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

All ranges disclosed herein are inclusive of the recited endpoint and independently combinable (for example, the range of “from 2 mg to 10 mg” is inclusive of the endpoints, 2 mg and 10 mg, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9 to 1.1. Other meanings of “about” may be apparent from the context, such as rounding off, so, for example “about 1” may also mean from 0.5 to 1.4.

The compounds of formula I, formula II, or formula III, as described herein, may be used in pharmaceutically acceptable salt forms or in free (i.e. non-salt) form. Salt forms can be obtained by treating the free form with an acid or base. Of interest are the pharmaceutically acceptable acid and base addition salts, which are meant to comprise the therapeutically active non-toxic acid and base addition salt forms that the compounds are able to form. The pharmaceutically acceptable acid addition salts of the compounds of formula I, formula II or formula III can conveniently be obtained by treating the free form with such appropriate acid. Appropriate acids comprise, for example, inorganic acids such as hydrohalic acids, such as hydrobromic acid, or in particular hydrochloric acid; or sulfuric, nitric, phosphoric and the like acids; or organic acids such as, for example, acetic, propanoic, hydroxyacetic, lactic, pyruvic, oxalic, malonic, succinic, maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. The compounds of formula I may also be converted into the pharmaceutically acceptable metal or amine addition salt forms by treatment with appropriate organic or inorganic bases. Appropriate base salt forms comprise, for example, the ammonium salts, the alkali and earth alkaline metal salts, e.g. the lithium, sodium or potassium salts; or the magnesium or calcium salts; salts with organic bases, e.g. the benzathine, N-methyl-D-glucamine, hydrabamine salts, and salts with amino acids such as, for example, arginine, lysine, and the like. The term addition salt form is meant to also comprise any solvates that the compounds of formula I, formula II or formula III, as well as the salts thereof, may form. Such solvates are, for example, hydrates, alcoholates, e.g. ethanolates, and the like.

The present disclosure provides methods of treating HCV in a patient comprising administering to the patient an effective amount of:
a compound of formula I (simeprevir, “SMV”):

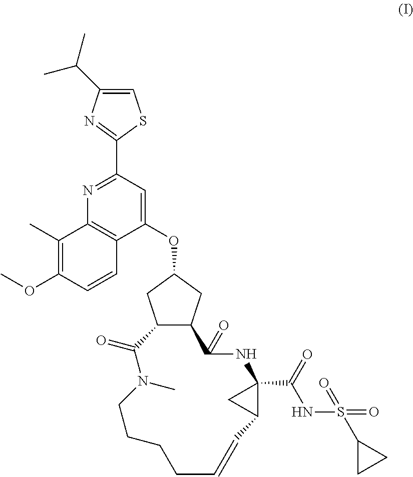 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]

or a pharmaceutically acceptable salt thereof,
a compound of formula II (daclatasvir, “DCV”):

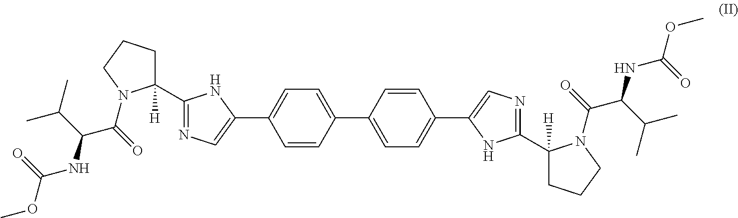

or a pharmaceutically acceptable salt thereof, and
a compound of formula III (sofosbuvir, “SOF”):

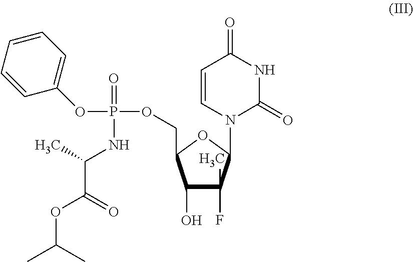

or a pharmaceutically acceptable salt thereof;
   wherein said administration terminates after a period of time that is 6, 7, 8, 9, 10, 11, or 12 weeks. In some embodiments, said administration terminates after a period of time that is 12 weeks. In other embodiments, said administration terminates after a period of time that is less than 12 weeks, for example, 6, 7, 8, 9, 10, or 11 weeks. Patients who can be treated using the described methods are preferably human. Other warm-blooded animals can also be treated.

Simeprevir has been described as an HCV NS3/4A protease inhibitor. It can be prepared according to methods known in the art, for example, those methods described in WO 2007/014926. A preferred form of simeprevir is simeprevir sodium salt.

Daclatasvir has been described as an HCV NS5A inhibitor. It can be prepared according to methods known in the art, for example, those methods described in WO 2008/021927. A prefered form of daclatasvir is daclatasvir dihydrochloride.

Sofosbuvir has been described as an HCV RNA polymerase NS5B inhibitor. It can be prepared according to methods known in the art, for example, those methods described in WO 2008/121634.

As used herein, “effective amount” refers to the amount of the compounds of formulas I, II, and III, or any pharmaceutically acceptable salts thereof, that elicits the biological or medicinal response in a tissue system (e.g., blood, plasma, biospy) or warm-blooded animal (e.g., human), that is being sought by a health care provider, which includes alleviation of the symptoms of the disease being treated.

The present disclosure is also directed to a combination comprising simeprevir (a compound of formula I), or a pharmaceutically acceptable salt thereof; daclatasvir (a compound of formula II), or a pharmaceutically acceptable salt thereof; and sofosbuvir (a compound of formula III), pharmaceutically acceptable salt thereof, for use in an HCV treatment regime that terminates after a period of time that is 12 weeks or less than 12 weeks, for example, 6, 7, 8, 9, 10, or 11 weeks.

Preferably, the administration of the compounds of formulas I, II, and III, or any salt form(s) thereof, terminates after a period of time that is less than 12 weeks, for example, 6, 7, 8, 9, 10, or 11 weeks. In preferred embodiments, the administration terminates after a period of time that is 6 weeks. In other embodiments, the administration terminates after a period of time that is 8 weeks.

In some embodiments, the patients treated according to the described methods will not have decompensated liver disease. In these embodiments, the administration preferably terminates after a period of time that is less than 12 weeks, for example, 6, 7, 8, 9, 10, or 11 weeks. Preferably, in these embodiments, the administration terminates after a period of time that is 6 weeks or 8 weeks.

In alternative embodiments, the administration of the compounds of formulas I, II, and III, or any salt form(s) thereof, terminates after a period of time that is 12 weeks.

In some embodiments, the patients treated according to the described methods will have decompensated liver disease (e.g., liver function is insufficient, Child-Pugh A, Child-Pugh B) prior to initiation of the treatment. In these embodiments, the administration preferably terminates after a period of time that is 12 weeks.

The treatments disclosed herein include the administration of the compounds of formulas I, II, and III, or any salt form(s) thereof, and does not include administering interferon, for example, PEGylated interferon, during the treatment period.

In some embodiments, the described methods do not include administration of ribavirin during the treatment period. In other embodiments, the described methods further include administration of ribavirin during the treatment period.

The described methods can be used to treat an HCV infection in a patient. Prior to initiation of treatment, the HCV infection can be diagnosed using methods known in the art, for example, by testing an HCV RNA level present in a biological sample taken from the patient, for example, a blood, plasma, or biopsy sample. Patients who can be treated using the described methods will have a quantifiable HCV RNA level greater than the lower limit of quantification (“LLOQ”) of the Roche COBAS Ampliprep/COBAS Taqman™ HCV Quantitative Test v2.0 (Roche Diagnostics, Indianapolis, Ind.). The LLOQ of that assay is 15 IU/mL.

Patients treated according to the methods of the disclosure can be “treatment naïve” patients. As used herein, “treatment naïve” refers to the patient not having previously received treatment with any drug—investigational or approved—for HCV infection.

Alternatively, patients treated according to the methods of the disclosure can be “treatment experienced.” As used herein, “treatment experienced” refers to a patient who has had at least one previous course of a non-direct-acting antiviral agent (“DAA”), interferon-based HCV therapy, with or without ribavirin. Preferably, the last dose in this previous course occurred at least two months prior to implementing a treatment regime according to the present disclosure.

HCV infections that can be treated according to the disclosed methods include HCV genotype 1 infections, for example, HCV genotype 1a infections and genotype 1b infections. Other infections that can be treated using the disclosed methods include HCV genotype 4 infections. HCV genotyping can be performed using methods known in the art, for example, VERSANT™ HCV Genotype 2.0 Assay Line Probe Assay (LiPA).

The methods described herein may be used to treat HCV infections that are comorbid with other liver diseases. For example, the HCV infection can be comorbid with liver fibrosis, cirrhosis, Child-Pugh A (mild hepatic impairment), or Child-Pugh B (moderate hepatic impairment), prior to initiation of the treatment.

Patients who can be treated according to the methods of the disclosure, in addition to having an HCV infection prior to initiation of the treatment, can also suffer from liver fibrosis prior to initiation of the treatment. For example, a patient can also suffer from liver fibrosis characterized by methods known in the art, such as a FibroSURE™ score of less than or equal to 0.48 and an aspartate aminotransferase to platelet ratio index (APRI) score of less than or equal to 1.

Patients who can be treated according to the methods of the disclosure, in addition to having an HCV infection prior to initiation of the treatment, can also suffer from cirrhosis prior to initiation of the treatment. For example, a patient can also suffer from cirrhosis characterized by methods known in the art, such as a FibroSURE™ score of greater than 0.75 and an aspartate aminotransferase to platelet ratio index (APRI) score of greater than 2, prior to initiation of the treatment. Alternatively, the patient can also suffer from cirrhosis characterized by a METAVIR score F4, prior to initiation of the treatment.

Patients who can be treated according to the methods of the disclosure, in addition to having an HCV infection prior to initiation of the treatment, can also suffer from Child-Pugh A (mild hepatic impairment) prior to initiation of the treatment.

Patients who can be treated according to the methods of the disclosure, in addition to having an HCV infection prior to initiation of the treatment, can also suffer from Child-Pugh B (moderate hepatic impairment) prior to initiation of the treatment. Evidence of portal hypertension characterized by, for example, esophageal varices or hepatic venous pressure gradient (HVPG) greater than or equal to 10 mm Hg, can be present prior to initiation of the treatment.

Patients treated according to the methods of the disclosure will achieve sustained virologic response (SVR) for up to 4 weeks after termination of the administration of the compounds of formulas I, II, and III, or any pharmaceutical salts thereof (i.e., SVR4). As used herein, “SVR” refers to an HCV RNA level of less than LLOQ. In other embodiments, the patient will achieve SVR for up to 12 weeks after termination of the administration of the compounds of formulas I, II, and III, or any pharmaceutical salts thereof (i.e., SVR12). In still other embodiments, the patient will achieve SVR for up to 24 weeks after termination of the administration of the compounds of formulas I, II, and III, or any pharmaceutical salts thereof (i.e., SVR24).

In some aspects of the disclosure, SVR will be achieved after 4 weeks (SVR4), after 12 weeks (SVR12), and/or after 24 weeks (SVR24) in at least 80% of patients treated according to the described methods. For example, SVR will be achieved after 4 weeks (SVR4), after 12 weeks (SVR12), and/or after 24 weeks (SVR24) in greater than 90% of patients, or greater than 95% of patients, treated according to the described methods.

In some embodiments, at least 80% of patients, for example, greater than 90% or greater than 95% of patients, treated according to the described methods will have HCV RNA levels of less than LLOQ, when tested during the treatment period. For example, at least 80% of patients treated according to the described methods will have HCV RNA levels of less than LLOQ when tested during week 2 of the treatment period. In other embodiments, at least 80% of patients treated according to the described methods will have HCV RNA levels of less than LLOQ when tested during week 4 of the treatment period. In yet other embodiments, at least 80% of patients treated according to the described methods will have HCV RNA levels of less than LLOQ when tested during week 6 of the treatment period. In still other embodiments, at least 80% of patients treated according to the described methods will have HCV RNA levels of less than LLOQ when tested during week 8 of the treatment period.

In other embodiments, the described methods will result in a relatively lower percentage of patients being classified as “viral relapsers,” that is, patients who did not achieve SVR12 at the end of the treatment period and having an HCV RNA level of greater than LLOQ during week 24 after the end of the treatment period. In these embodiments, less than 10% of patients, preferably less than 5% or less than 2% of patients, will be classified as viral relapsers, when treated according to the described methods.

It is known in the art that patients infected with HCV genotype 1a containing the NS3 polymorphism Q80K demonstrate lower response rates to previously-described treatments, for example, treatments with simeprevir in combination with PEGylated interferon and ribavirin. Patients infected with HCV genotype 1a containing the NS3 polymorphism Q80K, treated according to the described methods, will achieve SVR for up to 4, 12, or 24 weeks after termination of the administration of the compounds of formulas I, II, and III, or any pharmaceutical salts thereof. In some aspects of the disclosure, SVR will be achieved using the described methods after 4 weeks (SVR4), after 12 weeks (SVR12), and/or after 24 weeks (SVR24) in at least 80% of patients infected with HCV genotype 1a containing the NS3 polymorphism Q80K. For example, SVR will be achieved using the described methods after 4 weeks (SVR4), after 12 weeks (SVR12), and/or after 24 weeks (SVR24) in greater than 90% of patients, or greater than 95% of patients, infected with HCV genotype 1a containing the NS3 polymorphism Q80K.

In preferred embodiments of the disclosure, when treated according to the present disclosure, patients will not exhibit a change from baseline in HCV nonstructural protein 3/4A (NS3/4A), NSSA, and NSSB during the period of administration.

In other embodiments, the methods of the disclosure will be safer than treatment methods previously described in the art, that is, the described methods will result in fewer or less severe adverse side effects in patients. In yet other embodiments, the methods of the disclosure will be better tolerated by patients, that is, patient compliance will be higher, when compared to treatment methods previously described in the art.

According to preferred methods of the disclosure, each of the compounds of formulas I, II, and III, or any pharmaceutical salts thereof are administered once per day during the period of administration. The compounds of formulas I, II, and III, or any pharmaceutical salts thereof, can be co-administered, sequentially administered, or administered substantially simultaneously. The compounds of formulas I, II, and III, or any pharmaceutical salts thereof can be administered substantially simultaneously, that is, taken within 30 minutes or less of each other, preferably 15 minutes or less of each other. In some embodiments, the compounds of formulas I, II, and III, or any pharmaceutical salts thereof are administered once per day, at approximately the same time each day. For example, the compounds of formulas I, II, and III, or any pharmaceutical salts thereof are administered within a time range of 4 hours of the original time of administration on the first day, that is, ±2 hours, preferably ±1 hour, more preferably ±30 minutes of the time on the original day.

In some embodiments, the compounds of formulas I, II, and III, or any pharmaceutical salts thereof are administered as separate oral capsules or oral tablets. In other preferred embodiments, the compounds of formulas I, II, and III, or any pharmaceutical salts thereof are administered in the forms that have already received regulatory approval in, for example, Europe, United States, or Japan.

All amounts mentioned in this disclosure refer to the free form (i.e. non-salt form). The above values represent free-form equivalents, i.e. quantities as if the free form would be administered. If salts are administered the amounts need to be calculated in function of the molecular weight ratio between the salt and the free form.

The daily doses described herein are calculated for an average body weight of about 70 kg and should be recalculated in case of paediatric applications, or when used with patients with a substantially diverting body weight.

Preferably, the compound of formula I (simeprevir), or a pharmaceutically acceptable salt thereof, is administered in an amount that is about 100 mg to about 200 mg per day. For example, the compound of formula I, or a pharmaceutically acceptable salt thereof, is administered in an amount that is about 100, 110, 120, 130, 140, 150, 160, 170, 180, 190, or 200 mg per day. In particularly preferred embodiments, the compound of formula I, or a pharmaceutically acceptable salt thereof, is administered in an amount that is about 150 mg per day.

Preferably, the compound of formula II (daclatasvir), or a pharmaceutically acceptable salt thereof, is administered in an amount that is about 20 mg to about 100 mg per day. For example, the compound of formula II, or a pharmaceutically acceptable salt thereof, is administered in an amount that is about 20, 30, 40, 50, 60, 70, 80, 90, or 100 mg per day. In particularly preferred embodiments, the compound of formula II, or a pharmaceutically acceptable salt thereof, is administered in an amount that is about 60 mg per day.

Preferably, the compound of formula III (sofosbuvir), or a pharmaceutically acceptable salt thereof, is administered in an amount that is about 200 mg to about 600 mg per day. For example, the compound of formula III, or a pharmaceutically acceptable salt thereof, is administered in an amount that is about 200, 225, 250, 275, 300, 325, 350, 375, 400, 425, 450, 475, 500, 525, 550, 575, or 600 mg per day. In particularly preferred embodiments, the compound of formula III, or a pharmaceutically acceptable salt thereof, is administered in an amount that is about 400 mg per day.

The following examples are merely illustrative and are not intended to limit the disclosure to the materials, conditions, or process parameters set forth therein.

### EXAMPLES

### Materials and Methods

The compounds used in the treatment regimes are tablets/capsules that have already received regulatory approval (e.g. in Europe and/or the United States). Hence, it is preferred that the following amounts of active therapeutic agent are employed daily in the treatment regime: simeprevir (150 mg), daclatasvir (60 mg), sofosbuvir (400 mg). It will be understood that such amounts refer only to the weights of the non-salt moieties; if such actives are formulated in a certain salt form (e.g. simeprevir sodium salt, daclatasvir dihydrochloride), the net weight of that part will proportionately increase. Further, it will also be understood that the actives will be formulated into the relevant capsules, for example with (a) pharmaceutically acceptable carrier(s) and/or excipient(s)—in this respect, it is most preferred that the formulations (comprising such amounts of active) approved in Europe (and/or US, if applicable) are employed in the treatment regime.

The in vitro antiviral activity against HCV of described combinations can be tested in a cellular HCV replicon system based on Lohmann et al. (1999) Science 285:110-113, with the further modifications described by Krieger et al. (2001) Journal of Virology 75: 4614-4624 (incorporated herein by reference). This model, while not a complete infection model for HCV, is accepted as a robust and efficient model of autonomous HCV RNA replication The in vitro antiviral activity against HCV can also be tested by enzymatic tests.

### Example I

### Methods

This is an open-label (patients and researchers are aware about the treatment patients are receiving) and multicenter (more than 1 hospital or medical school team work) study regarding the combination of simeprevir, daclatasvir, and sofosbuvir. The study consists of a screening phase (6 weeks); an open-label treatment thase (6 weeks for Arm A and 8 weeks for Arm B; and a post-treatment follow-up phase (until 24 weeks after end of study treatment). Using a staggered approach, all eligible patients are assigned to 1 of the 2 arms, according to their level of fibrosis. The study is for males and females aged 18 to 70 years and is non-randomized.

Arm A consists of chronic HCV genotype 1 infected patients with early stages of liver fibrosis). Patients receive a combination therapy of simeprevir 150 mg capsule, daclatasvir 60 mg capsule, and sofosbuvir 400 mg capsule, once daily for 6 weeks.

Arm B consists of chronic HCV genotype 1 infected patients with cirrhosis. Patients receive a combination therapy of simeprevir 150 mg capsule, daclatasvir 60 mg capsule and sofosbuvir 400 mg capsule once daily for 8 weeks.

A sub-study is performed at a selected study site, where only patients who are eligible to participate in either Arm A/B and the sub-study are enrolled. Intra-hepatic and plasma HCV ribonucleic acid (RNA) levels, intra-hepatic, peripheral innate and adaptive immune responses during the treatment, are assessed in the sub-study.

### Eligibility

Inclusion Criteria: HCV genotype 1 infection and HCV RNA plasma level greater than (>) 10,000 international units per milliliter (IU/mL), both determined at screening.

Patients of Arm A will have evidence of early stages of liver fibrosis, defined by a FibroSURE score less than or equal to (≦) 0.48 and aspartate aminotransferase to platelet ratio index (APRI) score≦1

Patients of Arm B will have evidence of cirrhosis, defined by a FibroSURE score>0.75 and APRI score>2, or a previous (historical) biopsy documenting a METAVIR score F4. In addition, patients should have absence of esophageal varices or presence of small (grade 1) esophageal varices determined by upper gastrointestinal endoscopy, and absence of findings indicative of hepatocellular carcinoma in an ultrasonography.

Patients will be HCV treatment-naïve, defined as not having received treatment with any approved or investigational drug for chronic HCV infection.

Patients will be pegylated interferon (PegIFN) and ribavirin (RBV) eligible, defined as not having any contraindication to the use of PegIFN and RBV, in line with the prescribing information for each compound

Exclusion Criteria:

A. Main Study:

Patients will not have coinfection with HCV of another genotype than genotype 1 and/or human immunodeficiency virus (HIV) type 1 or 2 (positive HIV-1 or HIV 2 antibody test at screening).

Patients will not have any evidence of liver disease of non-HCV etiology. This includes, but is not limited to, acute hepatitis A infection, hepatitis B infection (hepatitis B surface antigen positive), drug- or alcohol-related liver disease, autoimmune hepatitis, hemochromatosis, Wilson's disease, alpha-1 antitrypsin deficiency, non-alcoholic steatohepatitis, primary biliary cirrhosis, or any other non-HCV liver disease considered clinically significant.

Patients will not have evidence of clinical hepatic decompensation or presence of grade 2/3 esophageal varices.

B. Sub-Study:

Patients will not have presence of coagulopathy (hemophilia) or hemoglobinopathy (including sickle cell disease, thalassemia).

Patients will not use of any anti-coagulant (for example, warfarin, heparin) or anti-platelet medications within 1 week of the screening visit.

### Results

| Outcome Measures | Timeframe | Description |
|---|---|---|
| Percentage of Patients | Baseline up to Week 6 | Patients who will have |
| With On-treatment | (Arm A) or Week 8 (Arm | HCV RNA <LLOQ |
| Virologic Response | B) | detectable or undetectable, |
| | | during treatment |
| Percentage of Patients | SVR4: 4 weeks after end | Patients who will have |
| With Sustained Virologic | of study drug treatment; | HCV RNA <LLOQ |
| Response at 4 Weeks | SVR24: 24 weeks after end | detectable or undetectable, |
| (SVR4) and 24 Weeks | of study drug treatment | 4 and 24 weeks after the |
| (SVR24) After end of | | actual end of study drug |
| Study Drug Treatment | | treatment. |
| Change from Baseline in | Screening up to Follow-up | Pre-treatment |
| HCV Nonstructural Protein | Week 24 | polymorphisms in the HCV |
| 3/4A (NS3/4A), NS5A and | | nonstructural protein 3/4A |
| NS5B Sequence in Patients | | (NS3/4A), NS5A and S5B |
| | | regions in all patients and |
| | | relevant changes in the |
| | | HCV NS3/4A, NS5A and |
| | | NS5B regions will be |
| | | described. |
| Percentage of Patients | Screening up to Follow-up | Patients who achieve SVR |
| With or Without an NS3 | Week 24 | with or without an NS3 |
| Q80K Polymorphism at | | Q80K polymorphism at |
| Baseline Achieving SVR | | Baseline. |

LLOQ - refers to lower limit of quantification (15 IU/mL).

### Example 2

This is an open-label study regarding the combination of simeprevir, daclatasvir, and sofosbuvir in treatment naïve patients and in patients who have failed at least one previous course of PEGylated interferon, with or without ribavirin. The study will have three parts: screening phase (about 4 weeks) and open-label treatment phase (from week 4 to week 16) and follow-up phase (until 5 years after the actual end of study drug treatment. SVR12 will be evaluated.

Panel 1: Patients will have Child-Pugh A (mild hepatic impairment) with evidence of portal hypertension (confirmed by presence of esophageal varices or hepatic venous pressure gradient (HVPG) greater than or equal to 10 mm Hg. The patients will receive simeprevir (150 mg capsule), daclatasvir (60 mg tablet), and sofosbuvir (400 mg tablet) orally once daily for 12 weeks.

Panel 2: Patients will have Child-Pugh B (moderate hepatic impairment). The patients will receive simeprevir (150 mg capsule), daclatasvir (60 mg tablet), and sofosbuvir (400 mg tablet) orally once daily for 12 weeks.

Inclusion Criteria

Patients will have documented chronic Hepatitis C virus (HCV) infection evidenced by diagnosis of HCV more than (>) 6 months before the screening visit, either by detectable HCV ribonucleic acid (RNA), a HCV positive antibody or the presence of histological changes consistent with chronic hepatitis.

Patients will have HCV genotype 1 or 4 infection and HCV RNA plasma level greater than 10,000 international unit per milliliter (IU/mL).

Patients will have cirrhosis, which is defined as a FibroScan with a result of greater than 14.5 kilopascals (kPa) at screening.

Patients will be HCV treatment-naïve (patient has not received treatment with any approved or investigational drug for the treatment of HCV infection and HCV treatment-experienced patients.

Other patients will have had at least 1 documented previous course of a non-direct-acting antiviral agent (DAA), interferon (IFN)-based HCV therapy (with or without Ribavirin [RBV]). The last dose in this previous course will have occurred at least 2 months prior to Screening.

Patients will have decompensated liver disease:

Panel 1: Child Pugh A (mild hepatic impairment) with evidence of portal hypertension [confirmed by the presence of esophageal varices on gastroscopy or hepatic venous pressure gradient (HVPG) greater than or equal to (≧) 10 millimeter of mercury (mm Hg)].

Panel 2: Child-Pugh B (moderate hepatic impairment)

Exclusion Criteria:

Patients will not have co-infection with any HCV genotype.

Patients will not have co-infection with human immunodeficiency virus (HIV)-1 or -2 (positive HIV-1 or HIV-2 antibodies test at screening).

Patients will not have co-infection with hepatitis B virus (hepatitis B surface antigen [HBsAg] positive).

Patients will not have any evidence of liver disease of non-HCV etiology. This includes, but is not limited to, acute hepatitis A infection, drug- or alcohol-related liver disease, autoimmune hepatitis, hemochromatosis, Wilson's disease, alpha-1 antitrypsin deficiency, non-alcoholic steatohepatitis, primary biliary cirrhosis, or any other non-HCV liver disease considered clinically significant.

### Results

| Outcome Measures | Timeframe | Description |
|---|---|---|
| Percentage of Patients | SVR4: 4 weeks after end | Patients who will have |
| With Sustained Virologic | of study drug treatment; | HCV RNA <LLOQ |
| Response at 4 Weeks | SVR12: 12 weeks after | detectable or undetectable, |
| (SVR4) 12 Weeks | end of study drug treatment | 4, 12, and 24 weeks after |
| (SVR12) and 24 Weeks | SVR24: 24 weeks after end | the actual end of study |
| (SVR24) After end of | of study drug treatment | drug treatment. |
| Study Drug Treatment | | |
| Change from Baseline in | Screening up to Follow-up | Pre-treatment |
| HCV Nonstructural Protein | Week 36 | polymorphisms in the HCV |
| 3/4A (NS3/4A), NS5A and | | nonstructural protein 3/4A |
| NS5B Sequence in Patients | | (NS3/4A), NS5A and S5B |
| | | regions in all patients and |
| | | relevant changes in the |
| | | HCV NS3/4A, NS5A and |
| | | NS5B regions will be |
| | | described. |

### Example 3

This is an open-label study regarding the combination of simeprevir and daclatasvir in treatment-naïve patients and in treatment-naïve patients infected with HCV genotype 1b or genotype 4. Patients can be co-infected with human immunodeficiency virus (HIV type 1 or HIV type 2). The study will have three parts: screening phase (about 4 weeks) and open-label treatment phase (from week 4 to week 16) and follow-up phase (until 24 weeks after the actual end of study drug treatment. SVR24 will be evaluated.

Arm 1 will consist of treatment-naïve patients who have HCV genotype 1b infection with advanced fibrosis or compensated cirrhosis (METAVIR F3/F4). Staging of fibrosis will be based on a non-invasive method or liver biopsy. Patients will receive simeprevir 150 mg (capsule) and daclatasvir 60 mg (capsule), once daily for 12 weeks.

Arm 2 will consist of treatment-naïve patients who have HCV genotype 1b infection with mild to moderate fibrosis (METAVIR F0-F2). Patients will receive simeprevir 150 mg (capsule) and daclatasvir 60 mg (capsule), once daily for 12 weeks.

Arm 3 will consist of treatment-naïve patients who have HCV genotype 4 infection with or without compensated cirrhosis (METAVIR F0-F4). Staging of fibrosis will be based on a non-invasive method or liver biopsy. Patients will receive simeprevir 150 mg (capsule) and daclatasvir 60 mg (capsule), once daily for 12 weeks.

### Eligibility

Main Selection Criteria:

Inclusion Criteria:

Patients will have chronic HCV genotype 1b or genotype 4 infection confirmed at screening of an HCV RNA level greater than 10,000 IU/mL.

Patients will have documented fibrosis stage by either shear wave elastography (Fibroscan (F3>9.6 kPa; cirrhosis≧14.6 kPa) within 6 months or less before screening or between screening and Day 1 or a liver biopsy documenting METAVIR F0-F3 (within 24 months before screening) or F4 (at any previous time).

Patients will be treatment-naïve, i.e., have not received prior treatment for HCV with any approved or investigational drug.

Exclusion Criteria:

Patients having hepatocellular carcinoma will be ruled out in patients with cirrhosis.

Patients who have HCV genotype 1b infection will be excluded if they have coinfection with HCV of a genotype other than genotype 1b or have genetic variants coding for the NSSA-Y93H and/or L31MN amino acids substitutions.

Patients who have HCV genotype 4 will be excluded if they have co-infection with HCV of a genotype other than genotype 4.

Patients will be excluded, regardless of their genotype, if they have evidence of currect or previous episodes of hepatic decompensation, acute liver disease, or chronic liver disease of a non-HCV etiology, or hepatitis A or B co-infection.

Coinfection with Human Immunodeficiency Virus (Type 1 or Type 2)

Inclusion Criteria:

Patients will have HCV genotype 1b or genotype 4 and will be coinfected with HIV-1 or HIV-2.

Patients can either be receiving Highly Active Antiretroviral Therapy (cART) or not receiving cART (i.e., never received treatment or currectly not on treatment) at the time of screening.

Patients receiving cART will be taking a stable cART regimen (for at least 4 consecutive weeks prior to screening) and have a plasma HIV RNA level less than 50 copies/mL (for at least 24 consecutive weeks prior to screening) and CD4+ cell count greater than 250 cells/μL.

Patient not receiving cART will have a CD4+ cell count greater than 500 cells/μL, a plasma HIV RNA level less than 100,000 copies/mL at screening, and should be unlikely to require antiretroviral (ARV) therapy for the next 6 months.

### Results

| Outcome Measures | Timeframe | Description |
|---|---|---|
| Percentage of patients | SVR12: 12 weeks after end | Patients who will have |
| infected with HCV | of study drug treatment; | HCV RNA <LLOQ |
| genotype 1b and have | | detectable or undetectable, |
| advanced fibrosis or | | 12 weeks after the actual |
| compensated cirrhosis | | end of study drug |
| (METAVIR F3/F4) with | | treatment. |
| Sustained Virologic | | |
| Response at 12 weeks | | |
| (SVR12) after end of Study | | |
| Drug Treatment | | |
| Percentage of patients | SVR12: 12 weeks after end | Patients who will have |
| infected with HCV | of study drug treatment | HCV RNA <LLOQ |
| genotype 1b and have mild | | detectable or undetectable, |
| to moderate fibrosis | | 12 weeks after the actual |
| (METAVIR F0-F2) with | | end of study drug |
| Sustained Virologic | | treatment. |
| Response at 12 weeks | | |
| (SVR12) after end of Study | | |
| Drug Treatment | | |
| Percentage of patients | SVR12: 12 weeks after end | Patients who will have |
| infected with HCV | of study drug treatment | HCV RNA <LLOQ |
| genotype 1b (METAVIR | | detectable or undetectable, |
| F0-F4) with Sustained | | 12 weeks after the actual |
| Virologic Response at 12 | | end of study drug |
| weeks (SVR12) after end | | treatment. |
| of Study Drug Treatment | | |
| Percentage of patients | SVR12: 12 weeks after end | Patients who will have |
| infected with HCV | of study drug treatment | HCV RNA <LLOQ |
| genotype 4 (METAVIR | | detectable or undetectable, |
| F0-F4) with Sustained | | 12 weeks after the actual |
| Virologic Response at 12 | | end of study drug |
| weeks (SVR12) after end | | treatment. |
| of Study Drug Treatment | | |
| Percentage of Patients | SVR4: 4 weeks after end | Patients who will have |
| With Sustained Virologic | of study drug treatment; | HCV RNA <LLOQ |
| Response at 4 Weeks | SVR24: 24 weeks after end | detectable or undetectable, |
| (SVR4) and 24 Weeks | of study drug treatment | 4 and 24 weeks after the |
| (SVR24) after end of Study | | actual end of study drug |
| Drug Treatment | | treatment. |

### Example 4

Background: Interim analysis (IA) data from an ongoing Phase II open-label study assessing for the first time an all-oral regimen of simeprevir (SMV) (HCV NS3/4A protease inhibitor), in combination with daclatasvir (DCV) (HCV NS5A replication complex inhibitor) and sofosbuvir (SOF) (HCV nucleotide-analogue NS5B polymerase inhibitor) in chronic HCV genotype (GT)1/4-infected patients (pts) with decompensated liver disease, a population with a high medical need and limited therapeutic options.

Methods: HCV treatment-naïve or (peg)IFN±ribavirin treatment-experienced GT1/4-infected cirrhotic pts≧18 yrs with Child-Pugh (CP) score<7 (Class A) with evidence of portal hypertension or 7-9 (Class B) were enrolled. Pts received 12 wks of SMV (150 mg once daily [QD]), DCV (60 mg QD) and SOF (400 mg QD). Primary efficacy endpoint: sustained virologic response 12 wks after end of treatment (EOT, SVR12). Key secondary endpoints: SVR4, on-treatment failure, viral relapse, PK and safety.

Results: IA in 28 pts who received treatment (male 64.3%; median age 58.0 yrs; White 96.4%; GT1a/1b/4 71.4/25.0/3.6%; treatment-naïve/experienced 50/50%; Fibroscan score range 14.9-63.9 kPa; CP class A/B 19/9 pts). Baseline Q80K was present in 11/20 GT1a pts and no GT1b/GT4 pts. 25 pts reached EOT; 14 reached Wk4 follow-up. Virologic response rates (HCV RNA<15 IU/mL) for CP A/B were 89.5/33.3% at Wk2, 100/77.8% at Wk4 and 100/100% at Wks 8, 10 and 12. All pts with available data achieved SVR4 (CP A 12/12; CP B 2/2). Mean SMV exposure (AUC) was 1.3 fold higher in CP B vs CP A pts at Wk2, but individual pt exposures in the CP B group were within the range observed for CP A. Mean SMV exposures for both groups were comparable for Wk2 vs Wk8 (Table). Mean DCV and SOF exposures were each similar within groups and at Wk2 vs Wk8. Adverse events (AEs) occurred in 57.1% of pts (CP A/B 47.4/77.8%); all were Grade 1/2 at the time of analysis. One serious AE (sick sinus syndrome; unrelated to study medication) was reported 1 wk after EOT. Most common AEs: pruritus (3 events; CP A/B 1/2) and urinary tract infection (2 events; CP A/B 1/1). At the time of analysis, there were no deaths or discontinuations due to AEs. Laboratory abnormalities were generally Grade 1/2.

As this data indicates, the combination of SMV+DCV+SOF resulted in high on-treatment virologic response and SVR4 rates, and was safe and well tolerated in decompensated liver disease patients.

TABLE
Plamsa PK parameters

| | Week 2 | | | | Week 8 | | | |
|---|---|---|---|---|---|---|---|---|
| | Child-Pugh A | | Child-Pugh B | | Child-Pugh A | | Child-Pugh B | |
| Mean (SD) | (n = 19) | | (n = 9) | | (n = 18) | | (n = 9) | |
| Simeprevir | | | | | | | | |
| Cₘₐₓ (ng/mL) | 6976 | (4439) | 7951 | (4676) | 6029 | (3936) | 8841 | (5384) |
| AUC (h · ng/mL) | 113873 | (91579) | 144310 | (88836) | 98561 | (80028) | 176091 | (114832)* |
| Daclatasvir | | | | | | | | |
| Cₘₐₓ (ng/mL) | 1152 | (414) | 958 | (420) | 1072 | (313) | 854 | (427) |
| AUC (h · ng/mL) | 16022 | (6470) | 14250 | (7352) | 15574 | (5342) | 13530 | (6662) |
| Sofosbuvir | | | | | | | | |
| Cₘₐₓ (ng/mL) | 1571 | (738)** | 1743 | (1331) | 1276 | (856) | 1418 | (977) |
| AUC (h · ng/mL) | 2864 | (978)** | 3768 | (1817) | 2729 | (988) | 3769 | (1800) |

*n = 8;
**n = 18.
AUC, area under the concentration-time curve;
Cₘₐₓ, maximum plasma concentration

### Example 5

This study assessed the combination of SMV, SOF, and DCV for 12 weeks in HCV genotype 1- or 4-infected patients with decompensated liver disease. The primary endpoint was Sustained Virologic Response at 12 weeks (SVR12).

Patients were treatment naïve or treatment-experienced (prior peg-interferon with or without ribovarin) with chronic HCV genotype 1 or 4 infection with either decompensation or evidence of portal hypertension. Pharmacokinetic analysis performed at week 2 and week 8.

Subject requirements:
absence of hepatocellular carcinoma
absence of co-infection with any HCV genotype, hepatitis B, or HIV-1/-2
no prior treatment with a direct acting antiviral agent
total serum bilirubin≦3×upper limit of normal
eGFR (estimated glomerular filtration rate)≧30 mL/min (according to the CKD-EPI (chronic kidney disease epidemiology collaboration) equation
platelet count≧30,000/mm³
albumin≧2.5 g/dL
INR (international normalized ratio)≦2.5

Tables 1, 2, and 3 show the baseline demographics and liver function characteristics of the patient population for this study.

TABLE 1
Baseline demographics of the study participants

| | SMV + SOF + DCV | | | | | |
|---|---|---|---|---|---|---|
| | Child-Pugh A | | Child-Pugh B | | | |
| | (N = 19) | | (N = 21) | | Total (N = 40) | |
| Median age, years | 56.0 | (30-64) | 61.0 | (50-75) | 58.5 | (30-75) |
| (range) | | | | | | |
| Male, n (%) | 14 | (74) | 11 | (52) | 25 | (63) |
| White, n (%) | 18 | (95) | 21 | (100) | 39 | (98) |
| Black/African | 1 | (5) | 0 | | 1 | (3) |
| American, n (%) | | | | | | |
| Hispanic or Latino, n (%) | 13 | (68) | 10 | (48) | 23 | (58) |
| Body mass index, | 26.80 | (22.7-35.5) | 31.80 | (21.2-47.0) | 28.45 | (21.2-47.0) |
| median (range) | | | | | | |

TABLE 2
Baseline disease characteristics of the study participants

| | SMV + SOF + DCV | | | | | |
|---|---|---|---|---|---|---|
| | Child-Pugh A (N = 19) | | Child-Pugh B (N = 21) | | Total (N = 40) | |
| Median HCV RNA, | 5.78 | (4.8-6.8) | 5.60 | (4.0-6.7) | 5.72 | (4.0-6.8) |
| log₁₀ IU/mL (range) | | | | | | |
| Treatment- | 9 | (47) | 10 | (48) | 19 | (48) |
| experienced, n (%) | | | | | | |
| HCV genotype, n (%) | | | | | | |
| 1a | 15 | (79) | 11 | (52) | 26 | (65) |
| NS3 Q80K^{a} | 9 | (60) | 3 | (30) | 12 | (48) |
| 1b | 3 | (16) | 10 | (48) | 13 | (33) |
| 4 | 1 | (5) | 0 | | 1 | (3) |
| IL28B, n (%) | | | | | | |
| Non-CC | 15 | (79) | 18 | (86) | 33 | (83) |
| HCV RNA level | | | | | | |
| (IU/mL) | | | | | | |
| <400,000 | 8 | (42) | 11 | (52) | 19 | (48) |
| ≧400,000-≦800,000 | 4 | (21) | 0 | | 4 | (10) |
| >800,000 | 7 | (37) | 10 | (48) | 17 | (43) |
| <6,000,000 | 18 | (95) | 21 | (100) | 39 | (98) |

| | | | | | |
|---|---|---|---|---|---|
| ≧6,000,000 | 1 | (5) | 0 | 1 | (3) |

TABLE 3
Baseline liver function characteristics

| | SMV + SOF + DCV | | | | | |
|---|---|---|---|---|---|---|
| | Child-Pugh A (N = 19) | | Child-Pugh B (N = 21) | | Total (N = 40) | |
| Fibroscan score, kPa | 21.80 | (14.9-43.5) | 30.80 | (16.8-75.0) | 27.00 | (14.9-75.0) |
| (median, range) | | | | | | |
| MELD score, n (%) | | | | | | |
| <10 | 12 | (63) | 10 | (48) | 22 | (55) |
| ≧10-<15 | 7 | (37) | 9 | (43) | 16 | (40) |

| | | | | | |
|---|---|---|---|---|---|
| ≧15 | 0 | 2 | (10) | 2 | (5) |

| | | | | | |
|---|---|---|---|---|---|
| CP score, n (%) | | | | | |
| 5 | 14 | (74) | — | 14 | (35) |
| 6 | 5 | (26) | — | 5 | (13) |

| | | | | | |
|---|---|---|---|---|---|
| 7 | — | 9 | (43) | 9 | (23) |
| 8 | — | 8 | (38) | 8 | (20) |
| 9 | — | 4 | (19) | 4 | (10) |

### Results:

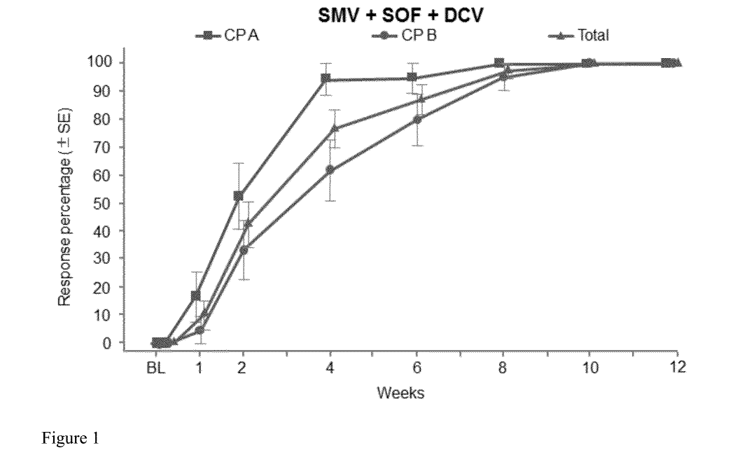

After 2 weeks of receiving a combination of SMV+SOF+DCV, 17/19 of the Child-Pugh A subjects exhibited HCV RNA of less than 15 IU/mL. All the Child-Pugh A subjects exhibited HCV RNA of less than 15 IU/mL after 4 weeks of treatment and after 12 weeks of treatment. Child-Pugh A subjects exhibited HCV RNA of less than 15 IU/mL at week 16 of the study (SVR4) and at week 24 of the study (SMV12). See also, FIG. 1.

After 2 weeks of receiving a combination of SMV+SOF+DCV, 11/21 of the Child-Pugh B subjects exhibited HCV RNA of less than 15 IU/mL. After 4 weeks of treatment, 19/21 of the Child-Pugh B subjects exhibited HCV RNA of less than 15 IU/mL. After 12 weeks of treatment, all of the Child-Pugh B subjects exhibited HCV RNA of less than 15 IU/mL. All of the Child-Pugh B subjects exhibited HCV RNA of less than 15 IU/mL at week 16 of the study (SVR4) and at week 24 of the study (SMV12). See also, FIG. 1.

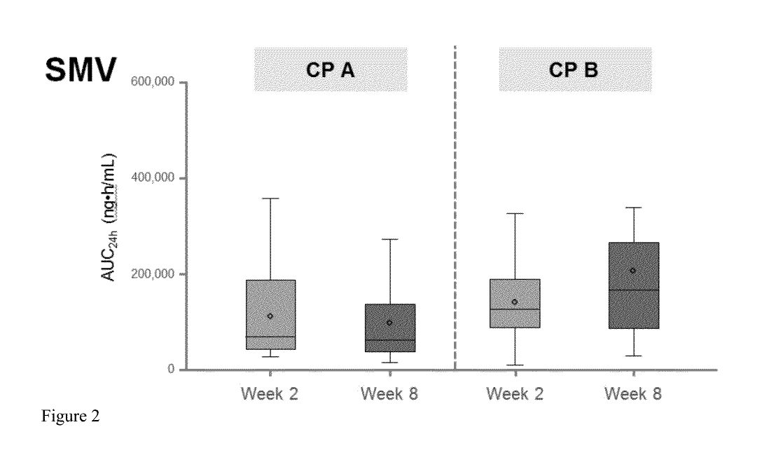

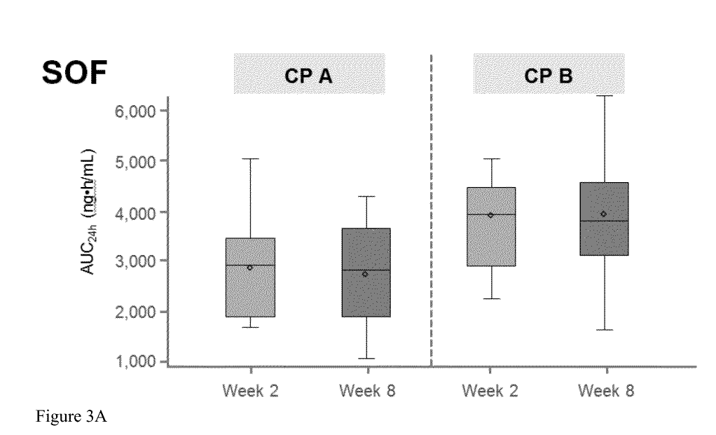

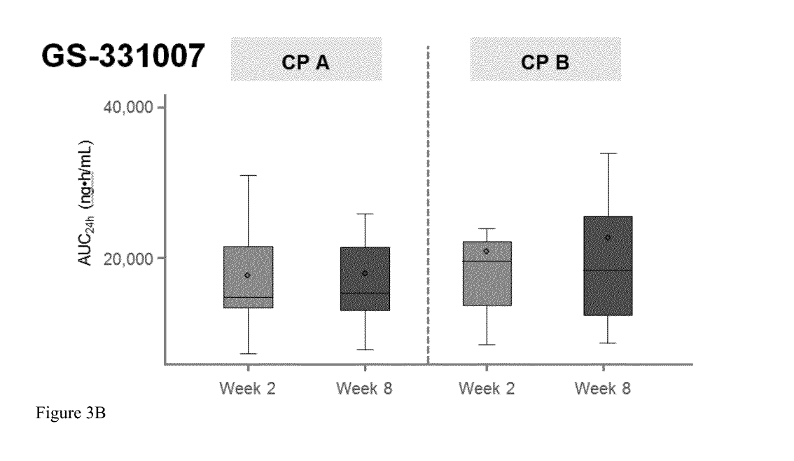

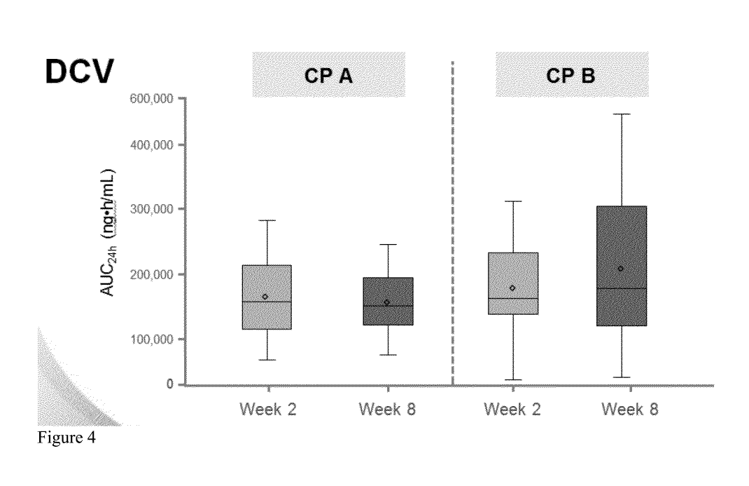

Pharmacokinetics of SMV, SOF, and DCV at week 2 and week 8 of treatment are depicted in FIGS. 2, 3A, 3B, and 4. As depicted in FIG. 2, the mean SMV exposure was 2.2-fold higher in Child-Pugh B subjects than in Child-Pugh A subjects. The mean SOF exposure was 1.4 fold higher in Child-Pugh B subjects than in Child-Pugh A subjects. See FIG. 3A. As depicted in FIG. 3B, the mean exposure of the SOF metabolite GS-331007 was similar in both Child-Pugh A and Child-Pugh B subjects. The mean exposure of DCV was similar in both Child-Pugh A and Child-Pugh B subjects. See FIG. 4.

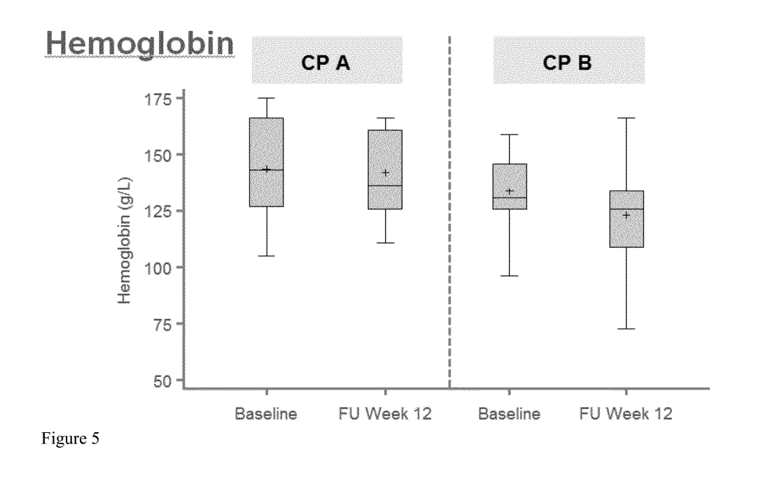

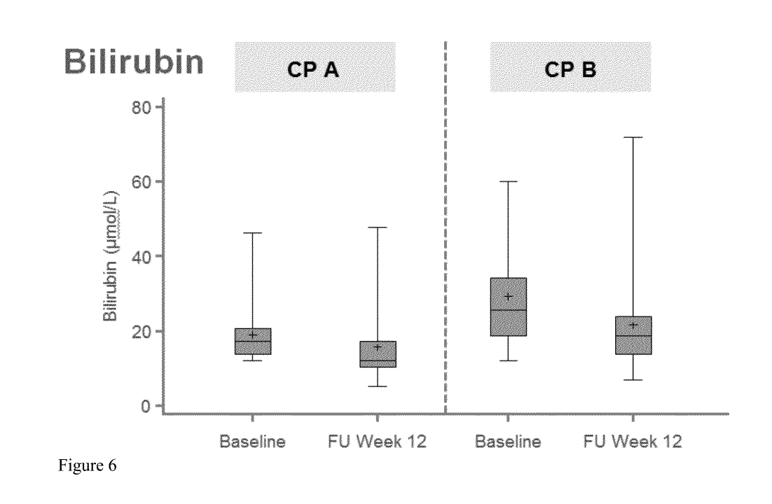

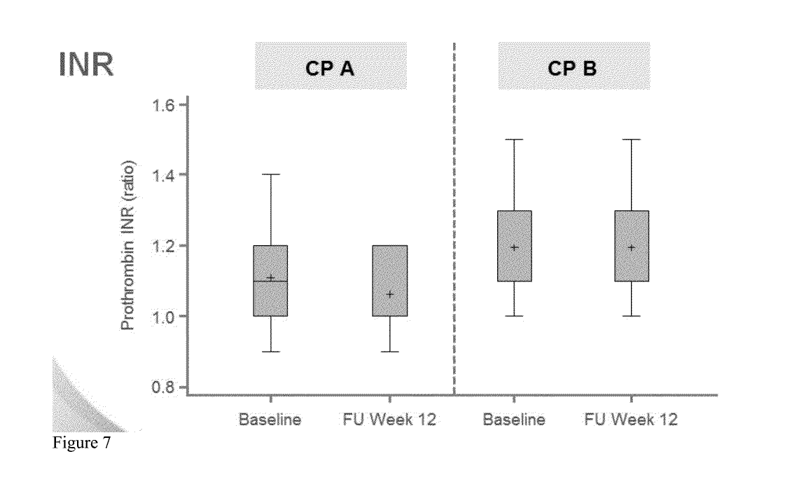

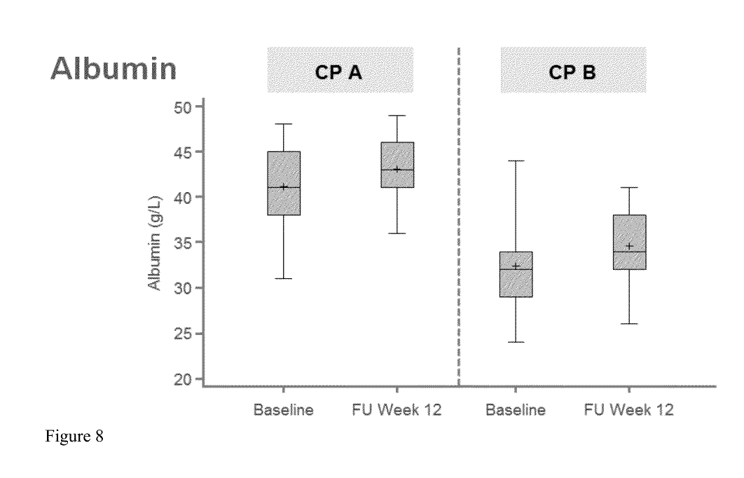

FIGS. 5-8 depict the median total hemoglobin, bilirubin, INR (international normalized ratio), and albumin prior to treatment with SMV+SOF+DCV and at week 12 of treatment with SMV+SOF+DCV.

Tables 4 and 5 depict adverse events observed during the treatment period. Table 6 depicts laboratory abnormalities observed during treatment.

TABLE 4
Summary of on-treatment adverse events (“AR”).

| | SMV + SOF + DCV | | |
|---|---|---|---|
| | Child-Pugh A | Child-Pugh B | |
| | (N = 19) | (N = 21) | Total (N = 40) |
| Any AE | 11 (58) | 16 (76) | 27 (68) |
| Grade 1/2 | 11 (58) | 15 (71) | 26 (65) |
| Grade 3/4 | 0 | 1 (5)^{a} | 1 (3) |
| Treatment-related AEs | | | |
| Possibly related to | 3 (16) | 7 (33) | 10 (25) |
| SMV | | | |
| Possibly related to | 1 (5) | 5 (24) | 6 (15) |
| SOF | | | |
| Possibly related to | 1 (5) | 5 (24) | 6 (15) |
| DCV | | | |
| Death | 0 | 0 | 0 |
| Serious AE | 0 | 1 (5)^{a} | 1 (3) |
| Early discontinuation | 0 | 0 | 0 |
| due to AE | | | |

TABLE 5
Most common on-treatment adverse events (≧2 subjects)

| | SMV + SOF + DCV | | |
|---|---|---|---|
| | Child-Pugh A | Child-Pugh B | |
| | (N = 19) | (N = 21) | Total (N = 40) |
| Pruritus | 1 (5) | 2 (10) | 3 (8) |
| Urinary tract infection | 1 (5) | 2 (10) | 3 (8) |
| Photosensitivity | 2 (11) | 1 (5) | 3 (8) |
| reaction | | | |
| Nausea | 1 (5) | 2 (10) | 3 (8) |
| Hepatic | 0 | 2 (10) | 2 (5) |
| encephalopathy | | | |
| Anemia | 2 (11) | 0 | 2 (5) |
| Insomnia | 0 | 2 (10) | 2 (5) |
| Irritability | 1 (5) | 1 (5) | 2 (5) |

TABLE 6
Laboratory abnormalities - worst WHO grade and treatment-emergent

| | SMV + SOF + DCV | | |
|---|---|---|---|
| | Child-Pugh A | | |
| | (N = 19) | Child-Pugh B (N = 21) | Total (N = 40) |
| Bilirubin^{a} | | | |
| Grade 3 | 2 (11) | 5 (24) | 7 (18) |
| Grade 4 | 0 | 2 (10) | 2 (5) |
| Glucose | | | |
| Grade 3 | 1 (5) | 1 (5) | 2 (5) |
| Grade 4 | 0 | 1 (5) | 1 (3) |
| Lipase | | | |
| Grade 3 | 0 | 1 (5) | 1 (3) |
| Grade 4 | 1 (5) | 0 | 1 (3) |
| Platelets | | | |
| Grade 3 | 0 | 3 (14) | 3 (8) |
| Grade 4 | 0 | 0 | 0 |

^{a}no concomitant increases in transaminases

### CONCLUSIONS

Treatment for 12 weeks with SMV, SOF, and DCV resulted in 100% SVR12. All 19 Child-Pugh A subjects and all 21 Child-Pugh B subjects achieved SVR12. High virologic response was observed regardless of Child-Pugh class (<7 or 8-9) or the presence of resistance-associated variants at baseline.

The combination of SMV, SOF, and DCV was generally safe and well tolerated. No deaths were observed and there were no discontinuations due to adverse events. A single, serious adverse event—gastrointestinal hemorrhage—was observed but it was unrelated to the treatment.

The study subjects will be observed at 5 years post-treatment. Liver function will be evaluated.

What is claimed:

1. A method of treating HCV in a patient having mild to moderate hepatic impairment comprising
administering to the patient having having mild to moderate hepatic impairment an effective amount of:
a compound of formula I: 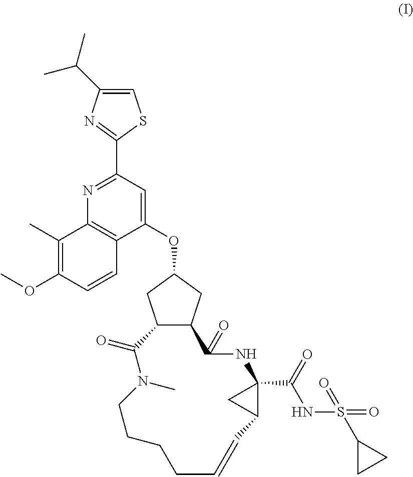 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]
or a pharmaceutically acceptable salt thereof,
a compound of formula II: 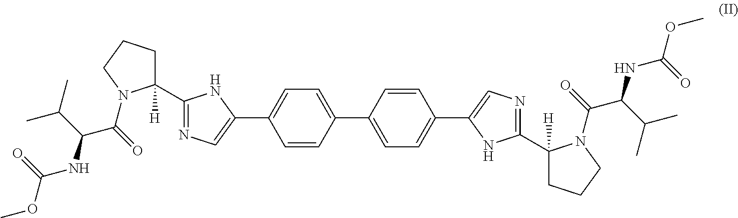
or a pharmaceutically acceptable salt thereof, and
a compound of formula III: 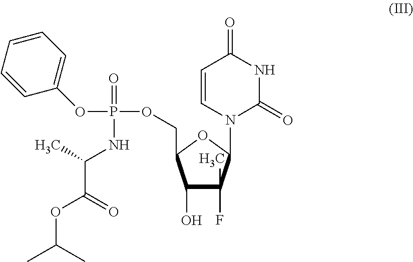
or a pharmaceutically acceptable salt thereof;
wherein said administration terminates after a period of time that is 6, 7, 8, 9, 10, 11, or 12 weeks.

2. The method of claim 1, wherein said period is 6 or 8 weeks.

3. The method of claim 1, wherein said period is 12 weeks.

4. The method of claim 1 that does not include administering interferon, PEGylated interferon, or ribavirin to said patient.

5. The method of claim 1, wherein the patient is a treatment naïve patient.

6. The method of claim 5, wherein the treatment naïve patient has not been previously treated with a direct acting antiviral agent.

7. The method of claim 1, wherein the HCV is HCV genotype 1.

8. The method of claim 7, wherein the HCV genotype 1 is HCV genotype 1a or HCV genotype 1b.

9. The method of claim 1, wherein the HCV is HCV genotype 4.

10. The method of claim 1, wherein the patient exhibits an HCV RNA plasma level greater than 10,000 IU/mL prior to initiation of the treatment.

11. The method of claim 1, wherein the patient suffers from liver fibrosis prior to the initiation of the treatment.

12. The method of claim 11, wherein the liver fibrosis is characterized by a FibroSURE score of less than or equal to 0.48 and an aspartate aminotransferase to platelet ratio index (APRI) score of less than or equal to 1, prior to initiation of the treatment.

13. The method of claim 1, wherein the patient suffers from cirrhosis prior to the initiation of the treatment.

14. The method of claim 13, wherein the cirrhosis is characterized by a FibroSURE score of greater than 0.75 and an aspartate aminotransferase to platelet ratio index (APRI) score of greater than 2, prior to initiation of the treatment.

15. The method of claim 13, wherein the cirrhosis is characterized by a METAVIR score F4, prior to initiation of the treatment.

16. The method of claim 1, wherein the patient suffers from Child-Pugh A (mild hepatic impairment), prior to initiation of the treatment.

17. The method of claim 1, wherein the patient suffers from Child-Pugh B (moderate hepatic impairment).

18. The method of claim 17, wherein the patient suffers from Child Pugh B with evidence of portal hypertension (evidenced by esophageal varices or hepatic venous pressure gradient (HVPG) greater than or equal to 10 mm Hg), prior to initiation of the treatment.

19. The method of claim 1, wherein the patient achieves sustained virologic response with an HCV RNA level of less than LLOQ for up to 24 weeks after termination of said administration.

20. The method of claim 1, wherein the patient achieves sustained virologic response with an HCV RNA level of less than LLOQ for up to 12 weeks after termination of said administration.

21. The method of claim 1, wherein the patient achieves sustained virologic response with an HCV RNA level of less than LLOQ for up to 4 weeks after termination of said administration.

22. The method of claim 1, wherein the patient does not exhibit a change from baseline in HCV nonstructural protein 3/4A (NS3/4A), NS5A, and NS5B during the period of administration.

23. The method of claim 1, wherein the patient exhibits an NS3 Q80K polymorphism prior to initiation of the treatment.

24. The method of claim 1, wherein the compounds, or salts thereof, are each administered once per day during the period of administration.

25. The method of claim 1, wherein the compounds, or salts thereof, are administered substantially simultaneously.

26. The method of claim 1, wherein the compound of formula I, or a pharmaceutically acceptable salt thereof, is administered in an amount that is about 100 mg to about 200 mg per day.

27. The method of claim 1, wherein the compound of formula II, or a pharmaceutically acceptable salt thereof, is administered in an amount that is about 20 mg to about 100 mg per day.

28. The method of claim 1, wherein the compound of formula III, or a pharmaceutically acceptable salt thereof, is administered in an amount that is about 200 mg to about 600 mg per day.
